(19) United States
(12) Patent Application Publication
(10) Pub. No.: US 2009/0143624 A1
(43) Pub. Date: Jun. 4, 2009

(54) COMPOSITIONS OF AND PROCESSES FOR PRODUCING POLY(TRIMETHYLENE GLYCOL CARBONATE TRIMETHYLENE GLYCOL ETHER) DIOL

(75) Inventors: Robert DiCosimo, Chadds Ford, PA (US); Neville Everton Drysdale, Newark, DE (US); Hari Babu Sunkara, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DuPont de Nemours and Company, Wilmington, DE (US)

(21) Appl. No.: 12/277,655

(22) Filed: Nov. 25, 2008

Related U.S. Application Data
(60) Provisional application No. 60/991,694, filed on Nov. 30, 2007.

(51) Int. Cl. C07C 43/10

(52) U.S. Cl. 568/595

(57) ABSTRACT

This invention relates to compositions of and processes for producing an unsubstituted or R-substituted poly(trimethylene glycol carbonate trimethylene glycol ether)diol. The processes use acidic ion exchange resins and include solvents.

### FIELD OF THE INVENTION

This invention relates to novel compositions of and processes for producing a poly(trimethylene glycol carbonate trimethylene glycol ether)diol. The processes use acidic ion exchange resins as catalysts and include solvents.

### BACKGROUND

There exists a need to produce dihydroxy-terminated materials. The materials described herein, poly(trimethylene glycol carbonate trimethylene glycol ether)diol, can be used in a number of applications, including but not limited to biomaterials, engineered polymers, personal care materials, coatings, lubricants and polycarbonate/polyurethanes (TPUs).

As described in Ariga et al., Macromolecules 1997, 30, 737-744 and in Kricheldorf et al., J. Macromol. Sci.—Chem A26(4), 631-644 (1989), in the cationic polymerization of TMC, the initiating agent becomes incorporated into the polymer ends.

### SUMMARY OF THE INVENTION

One aspect of the present invention is a poly(trimethylene glycol carbonate trimethylene glycol ether)diol oligomer of the structure.

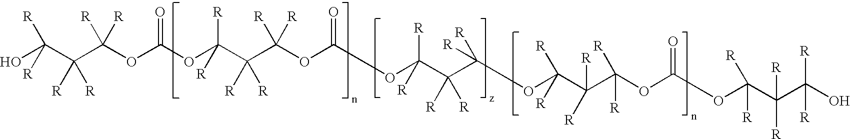

wherein z is an integer of about 1 to 10, particularly 1 to 7, more particularly 1 to 5; and n is an integer of about 2 to 100, particularly 2 to 50; and each R substituent is independently selected from the group consisting of H, C₁-C₂₀ alkyl, C₃-C₂₀ cyclic alkyl, C₅-C₂₅ aryl, C₆-C₂₀ alkaryl, and C₆-C₂₀ arylalkyl; and wherein each R substituent can optionally form cyclic structural groups with adjacent R substituents. Typically such cyclic structural groups are C₃-C₈ cyclic groups, e.g., cyclopropane, cyclobutane, cyclopentane, cyclohexane, cycloheptane, and cyclooctane.

Another aspect of the present invention is a process for making a poly(trimethylene glycol carbonate trimethylene glycol ether)diol oligomer of structure

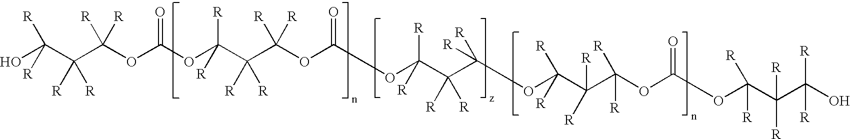

wherein z is an integer of about 1 to 10, particularly 1 to 7, more particularly 1 to 5;

n is an integer of about 2 to 100, particularly 2 to 50; and each R is independently selected from the group consisting of H, C₁-C₂₀ alkyl, C₃-C₂₀ cyclic alkyl, C₅-C₂₅ aryl, C₆-C₂₀ alkaryl, and C₆-C₂₀ arylalkyl; and wherein each R substituent can optionally form cyclic structural groups with adjacent R substituents; the process comprising: contacting trimethylene carbonate or an R-substituted trimethylene carbonate with an acidic ion exchange resin catalyst in the presence of a solvent at temperature of about 30 to 250 degrees Celsius to form a mixture comprising a poly(trimethylene glycol carbonate trimethylene glycol ether)diol oligomer composition.

### DETAILED DESCRIPTION

The present invention relates to a process to make poly(trimethylene glycol carbonate trimethylene glycol ether)diols from trimethylene carbonate (TMC, 1,3-dioxan-2-one) or a substituted trimethylene carbonate via elevated temperature (generally about 30 to 250 degrees Celsius) polymerization in the presence of a solvent utilizing an acidic ion exchange resin as a catalyst. This reaction can be represented by the Equation below:

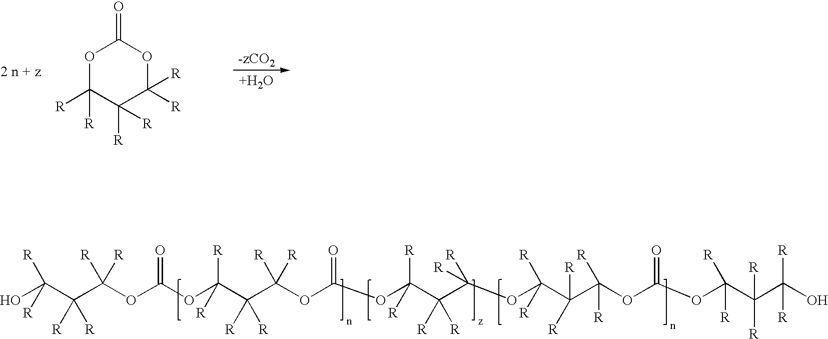

In the structure above, each R is independently selected from the group consisting of H, C₁-C₂₀ alkyl, particularly C₁-C₆ alkyl, C₃-C₂₀ cyclic alkyl, C₃-C₆ cyclic alkyl, C₅-C₂₅ aryl, particularly C₅-C₁₁ aryl, C₆-C₂₀ alkaryl, particularly C₆-C₁₁ alkaryl, and C₆-C₂₀ arylalkyl, particularly C₆-C₁₁ arylalkyl; and each R substituent can optionally form cyclic structural groups with adjacent R substituents. Typically such cyclic groups are C₃-C₈ cyclic structural groups, e.g., cyclopropane, cyclobutane, cyclopentane, cyclohexane, cycloheptane, and cyclooctane.

In the structure above, n is an integer of about 2 to 100, and more particularly about 2 to 50; and z is an integer of about 1 to about 10, particularly about 1 to 7, more particularly about 1 to 5.

When R is H in the structure above, the trimethylene carbonate (TMC) can be derived from, for example, 1,3-propanediol, or from poly(trimethylene carbonate).

Trimethylene carbonate is prepared by any of the various chemical or biochemical methods known to those skilled in the art. Chemical methods for the preparation of TMC include, but are not limited to, a) reacting 1,3-propanediol with diethylcarbonate in the presence of zinc powder, zinc oxide, tin powder, tin halide or an organotin compound at elevated temperature, b) reacting 1,3-propanediol and phosgene or bis-chloroformates to produce a polycarbonate intermediate that is subsequently depolymerized using heat and, optionally, a catalyst, c) depolymerizing poly(trimethylene carbonate) in a wiped film evaporator under vacuum, d) reacting 1,3-propanediol and urea in the presence of metal oxides, e) dropwise addition of triethylamine to a solution of 1,3-propanediol and ethylchloroformate in THF, and f) reacting 1,3-propanediol and phosgene or diethylcarbonate. Biochemical methods for the preparation of TMC include, but are not limited to, a) lipase catalyzed condensation of diethylcarbonate or dimethylcarbonate with 1,3-propanediol in an organic solvent, and b) lipase-catalyzed depolymerization of poly(trimethylene carbonate) to produce TMC. The 1,3-propanediol and/or trimethylene carbonate (TMC) can be obtained biochemically from a renewable source (“biologically-derived” 1,3-propanediol).

Preferably the 1,3-propanediol used as the reactant or as a component of the reactant will have a purity of greater than about 99%, and more preferably greater than about 99.9%, by weight as determined by gas chromatographic analysis.

The purified 1,3-propanediol preferably has the following characteristics:
(1) an ultraviolet absorption at 220 nm of less than about 0.200, and at 250 nm of less than about 0.075, and at 275 nm of less than about 0.075; and/or
(2) a CIELAB “b*” color value of less than about 0.15 (ASTM D6290), and an absorbance at 270 nm of less than about 0.075; and/or
(3) a peroxide composition of less than about 10 ppm; and/or
(4) a concentration of total organic impurities (organic compounds other than 1,3-propanediol) of less than about 400 ppm, more preferably less than about 300 ppm, and still more preferably less than about 150 ppm, as measured by gas chromatography.

The poly(trimethylene glycol carbonate trimethylene glycol ether)diol oligomer can be isolated using known methods.

The processes disclosed herein use an acidic ion exchange resin as a catalyst. These materials are available from a number of sources, and are generally added to the reactants to form a reaction mixture. As shown in the examples below, conveniently small amounts of these catalysts afford high conversion rates within about 25 hours.

Examples of the acidic ion exchange resins employed in the present embodiments include sulfonated tetrafluoroethylene copolymers, for example NAFION® NR50 (tetrafluoroethylene/perfluoro(4-methyl-3,6-dioxa-7-octene-1-sulfonic acid) copolymer, an ionomer available from DuPont, Wilmington, Del.), and DOWEX® 50WX8-200 (an ion-exchange resin consisting of poly(styrenesulfonic acid) crosslinked with divinylbenzene) available from Acros Organics N.V., Fair Lawn, N.J.

The processes disclosed herein use one or more solvents. Generally, any solvent can be used, as long as it is substantially non-reactive with the reactants and/or catalyst (i.e., the solvent doesn't react with the reactants to form undesired materials). Examples of solvents useful in the process described herein include but are not limited to toluene and hexane. As shown in the examples below, lower amounts of solvent generally provide for higher conversion rates.

The process described herein occurs at elevated temperature, generally about 30 to 250 degrees Celsius, and more particularly about 50 to 150 degrees Celsius. Once the reactants are added together, they may be mixed by any convenient method. The process can be done in batch, semi-batch or continuous mode, and generally take place in an inert atmosphere (i.e., under nitrogen).

Once the reactants have been contacted with the catalyst in the presence of one or more solvents, the reaction is allowed to continue for the desired time. Generally, at least 6 percent of the TMC polymerizes to give the desired poly(trimethylene glycol carbonate trimethylene glycol ether)diol after about 3 to 6 hours, with greater than about 75 percent conversion achieved within about 25 hours. As shown in the examples below, 100 percent conversion is easily achieved by the proper selection of solvent and catalyst, and amounts thereof.

Additionally, the desired level of polymerization, m, can be achieved by selection of solvent and catalyst, and amounts thereof. As shown in the examples below, the use of toluene and NAFION® NR50 affords a diol oligomer with an m of greater than about 0.5. In the present embodiments, n is an integer of about 2 to 100, and more specifically about 2 to 50; and z is an integer of about 1 to about 20, more specifically about 1 to 10.

The resulting novel poly(trimethylene glycol carbonate trimethylene glycol ether)diols can be separated from the unreacted starting materials and catalyst by any convenient method, such as filtration, including filtration after concentration.

The process disclosed herein allows for the degree of polymerization to be selected based on the solvent and/or catalyst chosen, and the amount of those materials used. This is advantageous as the materials resulting from the process can vary in properties including viscosity. The novel diol produced, wherein the term “oligomer” refers to materials with n less than or equal to 20, can find wide uses in products such as biomaterials, engineered polymers, personal care materials, coatings, lubricants and polycarbonate/polyurethanes (TPUs).

### EXAMPLES

The processes carried out in the following examples can be represented by the equation:

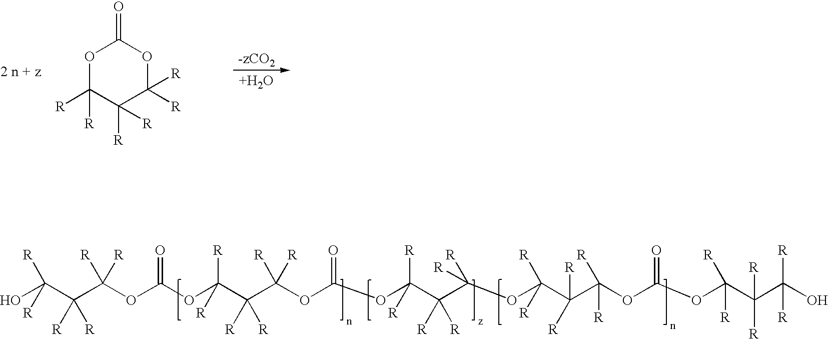

In the structure above, each R is independently selected from the group consisting of H, C₁-C₂₀ alkyl, particularly C₁-C₆ alkyl, C₃-C₂₀ cyclic alkyl, C₃-C₆ cyclic alkyl, C₅-C₂₅ aryl, particularly C₅-C₁₁ aryl, C₆-C₂₀ alkaryl, particularly C₆-C₁₁ alkaryl, and C₆-C₂₀ arylalkyl, particularly C₆-C₁₁ arylalkyl; and each R substituent can optionally form cyclic structural groups with adjacent R substituents. Typically such cyclic structural groups are C₃-C₈ cyclic structural groups, e.g., cyclopropane, cyclobutane, cyclopentane, cyclohexane, cycloheptane, and cyclooctane.

In the structure above, n is an integer of about 2 to 100, and more particularly about 2 to 50; and z is an integer of about 1 to about 10, particularly about 1 to 7, more particularly about 1 to 5.

### Examples 1-3

These examples describe the effect of various amounts of Nafion® NR50 ion exchange resin used as a catalyst on the production of poly(trimethylene glycol carbonate trimethylene glycol ether)diol. Toluene was used as the solvent, and the reactions were run at 100 degrees Celsius.

Trimethylene carbonate (10.00 g, 0.098 mol) and toluene (25 mL) were placed in three round bottomed flasks equipped with stirrers, reflux condensers and under nitrogen. To the first flask 0.5 g of Nafion® NR50 was added, to the second flask 1.0 g of Nafion® NR50 was added and to the third flask 2.00 g of Nafion® NR50 was added. The flasks were placed in oil baths maintained at 100 degrees Celsius and stirred. Aliquots were withdrawn after ˜6 hours and ˜22 hours, concentrated at reduced pressure and analyzed via Proton NMR. The table below shows the tabulated results:

| | Nafion ® NR50 | Conversion | Conversion |
|---|---|---|---|
| Example | (g) | (6 Hr) (%) | (22 Hr) (%) |
| 1 | 0.50 | 96.03 | 100 |
| 2 | 1.00 | 100 | — |
| 3 | 2.00 | 100 | — |

Upon cooling to room temperature two phases were apparent. The phases were separated, and then concentrated at reduced pressure. The top phases contained only a small amount of material, 0.57 g for Example 1, 0.62 g for Example 2, and 0.58 g for Example 3. The majority of the polymers, water clear, were contained in the bottom phases.

NMR analyses of the bottom phases gave the following:

| | Nafion ® NR50 | Molecular |
|---|---|---|
| Example | (g) | Weight (Mw) |
| 1 | 0.50 | 5826 |
| 2 | 1.00 | 3184 |
| 3 | 2.00 | 2090 |

A reduction in catalyst levels increased the molecular weight of the resulting polymer, while increasing the number of ether linkages.

### Examples 4-6

These examples illustrate the effect of various amounts of Nafion® NR50 catalyst on the production of poly(trimethylene glycol carbonate trimethylene glycol ether)diol. Toluene is used as the solvent, and the reactions were run at 50 degrees Celsius.

Trimethylene carbonate (10.00 g, 0.098 mol) and toluene (25 mL) were placed in three round bottomed flasks equipped with stirrers, reflux condensers and under nitrogen. To the first flask 0.5 g of Nafion® NR50 was added, to the second flask 1.0 g of Nafion® NR50 was added and to the third flask 2.00 g of Nafion® NR50 was added. The flasks were placed in oil baths maintained at 50 degrees Celsius and stirred. Aliquots were withdrawn after ˜3.5 hours and ˜22 hours, concentrated at reduced pressure and analyzed via Proton NMR. The table below shows the tabulated results:

| | Nafion ® NR50 | Conversion | Conversion |
|---|---|---|---|
| Example | (g) | (3.5 Hr) (%) | (22 Hr) (%) |
| 4 | 0.50 | 8.93 | 79.53 |
| 5 | 1.00 | 33.53 | 100 |
| 6 | 2.00 | 100 | 100 |

Upon cooling to room temperature two phases were apparent. The phases were separated, and then concentrated at reduced pressure. The top phases contained only a small amount of materials. The bottom phases were analyzed via Proton NMR. The results are tabulated below:

| | Nafion ® NR50 | Molecular |
|---|---|---|
| Example | (g) | Weight, Mw |
| 4 | 0.50 | 9794 |
| 5 | 1.00 | 5847 |
| 6 | 2.00 | 3735 |

### Examples 7-8

These examples describe the effect of various concentrations of toluene on the production of poly(trimethylene glycol carbonate trimethylene glycol ether)diol.

Trimethylene carbonate (10.00 g 0.098 mol) and Nafion® NR 50 (2.0 g) were placed in two oven dried flasks equipped with a stirrer, reflux condenser and under nitrogen. Toluene (50 and 100 mL) was added separately to each flask. The flasks were placed and stirred in oil baths maintained at ˜100 degrees Celsius. Aliquots were withdrawn after ˜6 hours and ˜22 hours, concentrated at reduced pressure and analyzed via Proton NMR. The table below shows the tabulated results:

| | | Conversion | Conversion |
|---|---|---|---|
| Example | Toluene (mL) | (6 Hr) (%) | (22 Hr) (%) |
| 7 | 50 | ~100 | — |
| 8 | 100 | 98.3 | — |

NMRs analyses of the bottom phases gave the following:

| | | Molecular |
|---|---|---|
| Experiment | Toluene (mL) | Weight, Mw |
| 7 | 50 | 1875 |
| 8 | 100 | 1795 |

### Examples 9-11

These examples describe the effect of various amounts of Nafion® NR50 catalyst on the production of poly(trimethylene glycol carbonate trimethylene glycol ether)diol. Hexane was used as the solvent, and the reactions were run at 65 degrees Celsius.

Trimethylene carbonate (10.00 g, 0.098 mol) and hexane (25 mL) were placed in three round bottomed flasks equipped with stirrers, reflux condensers and under nitrogen. To the first flask 0.5 g of Nafion was added, to the second flask 1.0 g of Nafion was added and to the third flask 2.00 g of Nafion was added. The flasks were placed in oil baths maintained at 65 degrees Celsius and stirred. Aliquots were withdrawn after ˜6 hours and ˜21 hours, concentrated at reduced pressure and analyzed via Proton NMR. The table below shows the tabulated results:

| | Nafion ® NR50 | Conversion | Conversion |
|---|---|---|---|
| Example | (g) | (6 Hr) (%) | (22 Hr) (%) |
| 9 | 0.50 | 26.50 | 71.50 |
| 10 | 1.00 | 39.23 | 89.65 |
| 11 | 2.00 | 85.62 | ~100 |

Upon cooling to room temperature two phases were apparent. The phases were separated, concentrated at reduced pressure. The top phases contained only a small amount of material. The majority of the polymers, water clear, were contained in the bottom phases. The bottom phases were concentrated at reduced pressure and analyzed via Proton NMRs, the results of which are tabulated in the following table:

| | Nafion ® NR50 | Molecular |
|---|---|---|
| Example | (g) | Weight (Calc) |
| 9 | 0.50 | 2799 |
| 10 | 1.00 | 3030 |
| 11 | 2.00 | 2422 |

### Example 12

### Larger Scale Reaction

Trimethylene carbonate (110.00 g, 1.078 mol), toluene (275.0 mL) and Nafion® NR 50 (22.0 g) were placed in an oven dried round bottomed flask equipped with a reflux condenser and under nitrogen. The reaction mixture was placed in an oil bath maintained at 100 degrees Celsius. After ˜22 hours, the reaction was cooled to room temperature, in which two phases resulted. The top phase, toluene, was decanted off and the resulting material filtered from the Nafion®. The Nafion® was washed with methylene chloride chloride. The combined filtrate and methylene chloride wash were combined and concentrated at reduced pressure and then dried under vacuum at ˜70 degrees Celsius. The resulting water clear material gave a calculated molecular weight of ˜2194, with m of ˜2.075.

DSC runs were made on a TA Instruments Q2000 DSC, using a 10° C./min heating rate and an N₂ purge. The profile used was heat, cool and reheat from −90 to 100 degrees Celsius. The TGA runs were made on a TA Instruments Q5000 TGA, again using a 10 degrees Celsius/min heating rate and an N₂ purge.

DSC analyses of this material gave a Tg of −33 degrees Celsius. (second heat). Also, fluorine analyses of this material via Wickbold Torch combustion gave 12 ppm. Thermal analyses, TGAs, heating rate of 10 degrees Celsius per minute, showed the material to be quite thermally stable, as shown in the following table:

| | Weight Lost (Decomposition) | | |
|---|---|---|---|
| | Temp (degrees Celsius) | | |
| Example 12 | 10% | 50% | 90% |
| Under Air | 299.54 | 343.97 | 366.49 |
| Under | 303.76 | 342.14 | 365.90 |
| Nitrogen | | | |

### Example 13

A stock solution containing trimethylene chloride (136.0 g) and diluted to one liter with toluene was prepared, representing a 1.33 M solution.

### Example 14A

### Nafion® Catalyst Cycle: Number 1

The above stock solution (Example 13, 75 mL) was added, via syringe, to an oven dried 100 mL round bottomed flask equipped with a stirrer, reflux condenser and under nitrogen, containing Nafion® NR50 (2.0 g). The reaction mixture was placed in an oil bath maintained at 100 degrees Celsius. Aliquots were withdrawn over time, concentrated at reduced pressure and analyzed via NMR. After completion of the reaction, the reaction mixture was filtered and the recovered Nafion catalyst was washed with methylene chloride (2×˜50 mL).

### Example 14B

### Nafion® Catalyst Cycle: Number 2

The recovered catalyst was placed in an oven dried 100 mL RB flask equipped with a stirrer and under nitrogen. To this material was added the above stock solution (75 mL), via syringe. The reaction mixture was placed in an oil bath maintained at 100 degrees Celsius. Aliquots were withdrawn over time, concentrated at reduced pressure and analyzed via NMR. After completion of the reaction, the reaction mixture was filtered and the recovered Nafion® catalyst was washed with methylene chloride (2×˜50 mL).

### Examples 14C-L

### Nafion® Catalyst Cycles 3-12

The above procedure of Number 2 was followed for the continuing number of cycle and the materials analyzed via Proton NMRs, the results of which are tabulated in the following table:

| | Cycle | Time | Conversion | Calc. |
|---|---|---|---|---|
| Example | Number | (Hr) | (%) | Mo. Wt. |
| 14A | 1 | 22 | 100 | 2147 |
| 14B | 2 | 70.5 | 100 | 2455 |
| 14C | 3 | 22 | 100 | 3255 |
| 14D | 4 | 22 | 100 | 4026 |
| 14E | 5 | 22 | 100 | 4732 |
| 14F | 6 | 22 | 100 | 3383 |
| 14G | 7 | 72 | 100 | 2840 |
| 14H | 8 | 22 | 22 | 4232 |
| 14I | 9 | 22 | 100 | 3467 |
| 14J | 10 | 22 | 100 | 3259 |
| 14K | 11 | 22 | 100 | 4840 |
| 14L | 12 | 72 | 100 | 3409 |

What is claimed is:

1. A poly(trimethylene glycol carbonate trimethylene glycol ether)diol oligomer of the structure. 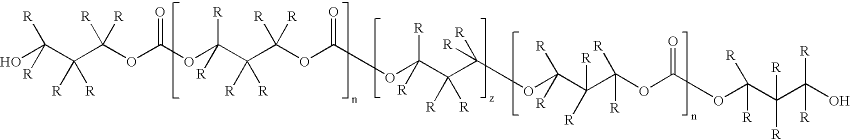
wherein z is an integer of about 1 to 10; n is an integer of about 2 to 100; and each R substituent is independently selected from the group consisting of H, C₁-C₂₀ alkyl, C₃-C₂₀ cyclic alkyl, C₅-C₂₅ aryl, C₆-C₂₀ alkaryl, and C₆-C₂₀ arylalkyl; and wherein each R substituent can optionally form cyclic structural groups with adjacent R substituents.

2. The poly(trimethylene glycol carbonate trimethylene glycol ether)diol oligomer of claim 1, wherein each R substituent is H.

3. The poly(trimethylene glycol carbonate trimethylene glycol ether)diol oligomer of claim 1, wherein z is about 1 to 7 and n is about 2 to 50.

4. A process for making a poly(trimethylene glycol carbonate trimethylene glycol ether)diol oligomer of structure 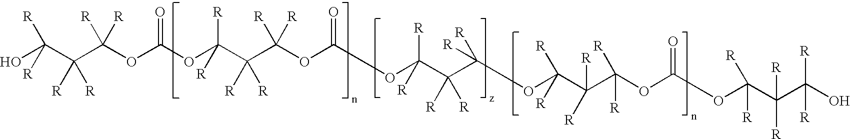
wherein z is an integer of about 1 to 10; n is an integer of about 2 to 100; and each R substituent is independently selected from the group consisting of H, C₁-C₂₀ alkyl, C₃-C₂₀ cyclic alkyl, C₅-C₂₅ aryl, C₆-C₂₀ alkaryl, and C₆-C₂₀ arylalkyl; and wherein each R substituent can optionally form cyclic structural groups with adjacent R substituents;
the process comprising: contacting trimethylene carbonate or an R-substituted trimethylene carbonate with an acidic ion exchange resin catalyst in the presence of a solvent at temperature of about 30 to 250 degrees Celsius to form a mixture comprising a poly(trimethylene glycol carbonate trimethylene glycol ether)diol oligomer composition.

5. The process of claim 4, wherein the solvent is substantially non-reactive with the trimethylene carbonate and the solvent.

6. The process of claim 5, wherein the non-reactive solvent is toluene or hexane.

7. The process of claim 4, wherein the acidic ion exchange resin catalyst is an ion-exchange resin comprising poly(styrenesulfonic acid) crosslinked with divinylbenzene.

7. The process of claim 4 wherein the acidic ion exchange resin catalyst is selected from the group consisting of tetrafluoroethylene/perfluoro(4-methyl-3,6-dioxa-7-octene-1-sulfonic acid) copolymers.

8. The process of claim 4, wherein the solid acid catalyst is tetrafluoroethylene/perfluoro(4-methyl-3,6-dioxa-7-octene-1-sulfonic acid) copolymer.

9. The process of claim 4 further comprising isolating the poly(trimethylene glycol carbonate trimethylene glycol ether)diol oligomer composition.

10. A poly(trimethylene glycol carbonate trimethylene glycol ether)diol oligomer made by the process of claim 4.
